(19) United States
(12) Patent Application Publication
(10) Pub. No.: US 2024/0353267 A1
(43) Pub. Date: Oct. 24, 2024

(54) THERMAL AND METAL DETECTION DEVICE

(71) Applicant: Robert Navarro, Gilbert, AZ (US)

(72) Inventor: Robert Navarro, Gilbert, AZ (US)

(21) Appl. No.: 18/136,592

(22) Filed: Apr. 19, 2023

(51) Int. Cl. G01K 1/14; G01K 1/08; G01K 1/20; G01N 33/20; H04N 23/23; H04R 1/02; H05K 5/00

(52) U.S. Cl. CPC G01K 1/14; G01K 1/08; G01K 1/20; G01N 33/20; H04N 23/23; H04R 1/02; H05K 5/0018; H05K 5/0026

(57) ABSTRACT

A thermal and metal detection device for attaching to a wearer and alerting the wearer of the thermal sensor when detecting a thermal signature of a person includes a housing including a front wall, a back wall, and a perimeter wall. An infrared sensor is coupled to the housing and detects the thermal signature of a person within a vicinity of the user. A metal sensor is coupled to the housing and detects a metal object within the vicinity of the user. A circuit board is electrically coupled to the infrared sensor and to the metal sensor. A plurality of securing members is coupled to the housing. Each of the securing members secures the housing to an object proximate to the user such that only one of the securing members selectively secures the housing to the object when in-use.

### CROSS-REFERENCE TO RELATED APPLICATIONS

Not Applicable

### STATEMENT REGARDING FEDERALLY SPONSORED RESEARCH OR DEVELOPMENT

Not Applicable

### THE NAMES OF THE PARTIES TO A JOINT RESEARCH AGREEMENT

Not Applicable

### INCORPORATION-BY-REFERENCE OF MATERIAL SUBMITTED ON A COMPACT DISC OR AS A TEXT FILE VIA THE OFFICE ELECTRONIC FILING SYSTEM

Not Applicable

### STATEMENT REGARDING PRIOR DISCLOSURES BY THE INVENTOR OR JOINT INVENTOR

Not Applicable

### BACKGROUND OF THE INVENTION

### (1) Field of the Invention

The disclosure relates to thermal sensor device and more particularly pertains to a new thermal sensor device for attaching to a wearer and alerting the wearer of the thermal sensor when detecting a thermal signature of a person.

### (2) Description of Related Art Including Information Disclosed Under 37 CFR 1.97 and 1.98

The prior art relates to thermal sensor devices and includes a variety of thermal sensor devices attaching to a user and being configured to detect thermal signature from a surrounding environment of the user. Known prior art does not include a thermal sensor device having a metal detector sensor being configured to detect metal objects within the surround environment of the user.

### BRIEF SUMMARY OF THE INVENTION

An embodiment of the disclosure meets the needs presented above by generally comprising a housing having an exterior surface and including a front wall, a back wall, and a perimeter wall. An infrared sensor is coupled to the housing and is configured to detect a thermal signature of a person within a vicinity of the user. A metal sensor is coupled to the housing and is configured to detect a metal object within the vicinity of the user. A circuit board is electrically coupled to the infrared sensor and to the metal sensor. A light emitter is electrically coupled to the circuit board is actuated by the circuit board to emit light when the infrared sensor is configured to detect the thermal signature of a person or when the metal sensor is configured to detect the metal object. A speaker is electrically coupled to the circuit board and is actuated by the circuit board the to emit an audible alarm when the infrared sensor is configured to detect the thermal signature of a person or when the metal sensor is configured to detect the metal object. A vibrating motor is electrically coupled to the circuit board and is actuated by the circuit board to emit vibrations when the infrared sensor is configured to detect the thermal signature of a person or when the metal sensor is configured to detect the metal object. A power supply is electrically coupled to the circuit board. A switch is electrically coupled to the circuit board and is actuated to turn on or off the infrared sensor and the metal sensor. A plurality of securing members is coupled to the housing. Each of the securing members is configured to secure the housing to an object proximate to the user such that only one of the securing members selectively secures the housing to the object when in-use.

There has thus been outlined, rather broadly, the more important features of the disclosure in order that the detailed description thereof that follows may be better understood, and in order that the present contribution to the art may be better appreciated. There are additional features of the disclosure that will be described hereinafter and which will form the subject matter of the claims appended hereto.

The objects of the disclosure, along with the various features of novelty which characterize the disclosure, are pointed out with particularity in the claims annexed to and forming a part of this disclosure.

### BRIEF DESCRIPTION OF SEVERAL VIEWS OF THE DRAWING(S)

The disclosure will be better understood and objects other than those set forth above will become apparent when consideration is given to the following detailed description thereof. Such description makes reference to the annexed drawings wherein:

FIG. 1 is a top isometric view of a thermal and metal detection device according to an embodiment of the disclosure.

FIG. 2 is a front view of an embodiment of the disclosure.

FIG. 3 is a back view of an embodiment of the disclosure.

FIG. 4 is a side view of an embodiment of the disclosure.

FIG. 5 is a cross-sectional view of an embodiment of the disclosure taken along Line 5-5 of FIG. 4.

FIG. 6 is an in-use view of an embodiment of the disclosure.

FIG. 7 is an in-use view of an embodiment of the disclosure.

FIG. 8 is an in-use view of an embodiment of the disclosure.

FIG. 9 is an in-use view of an embodiment of the disclosure.

FIG. 10 is a block diagram view of an embodiment of the disclosure.

### DETAILED DESCRIPTION OF THE INVENTION

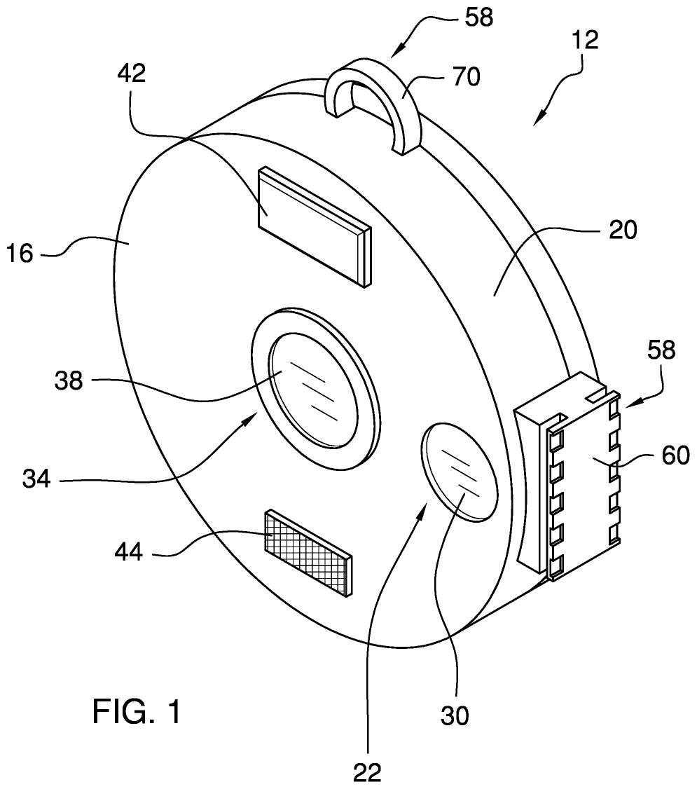

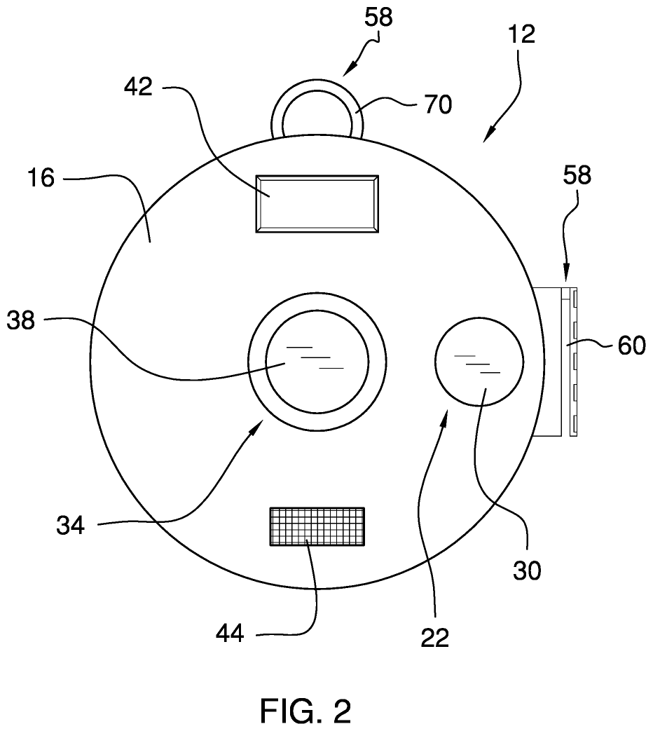

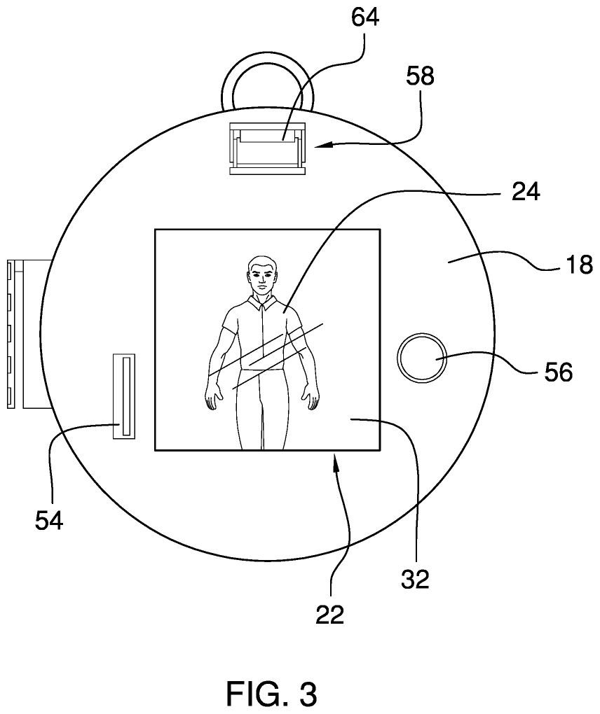

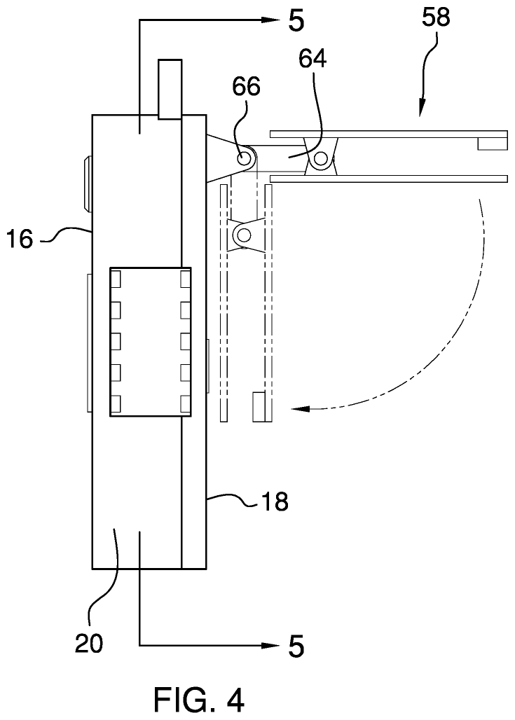

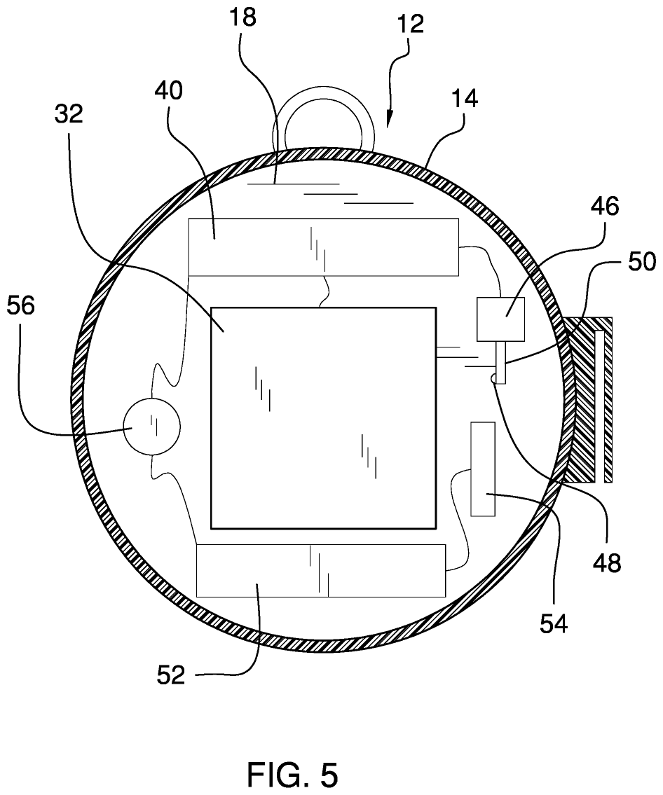

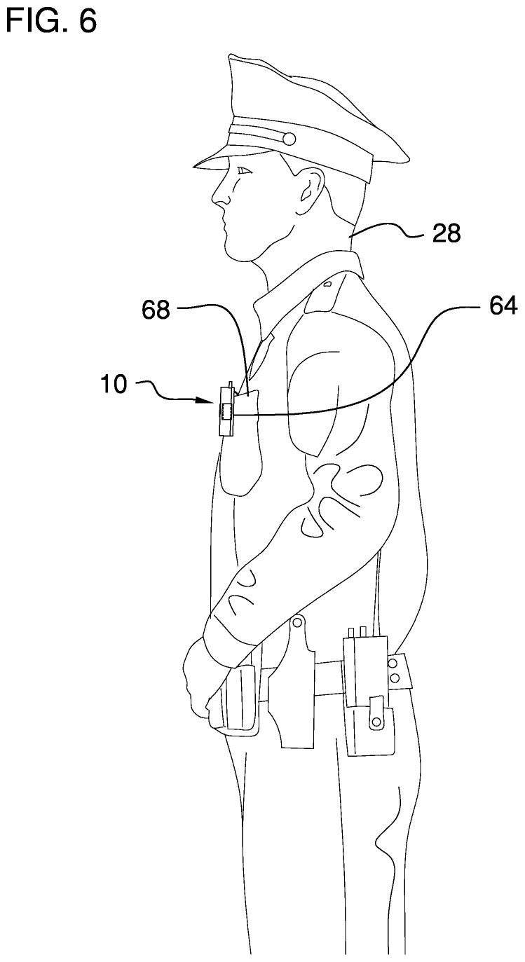

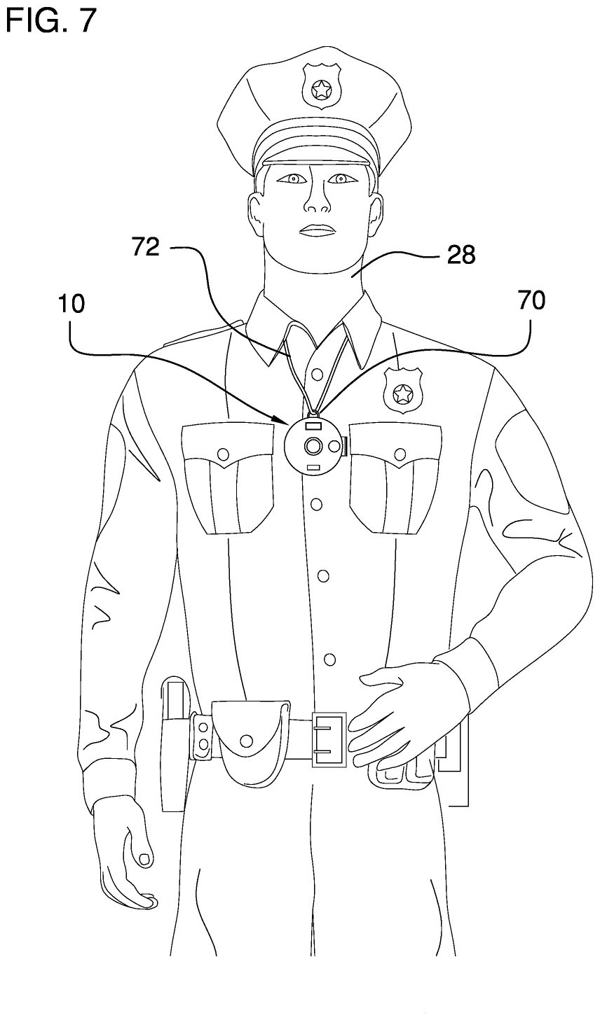

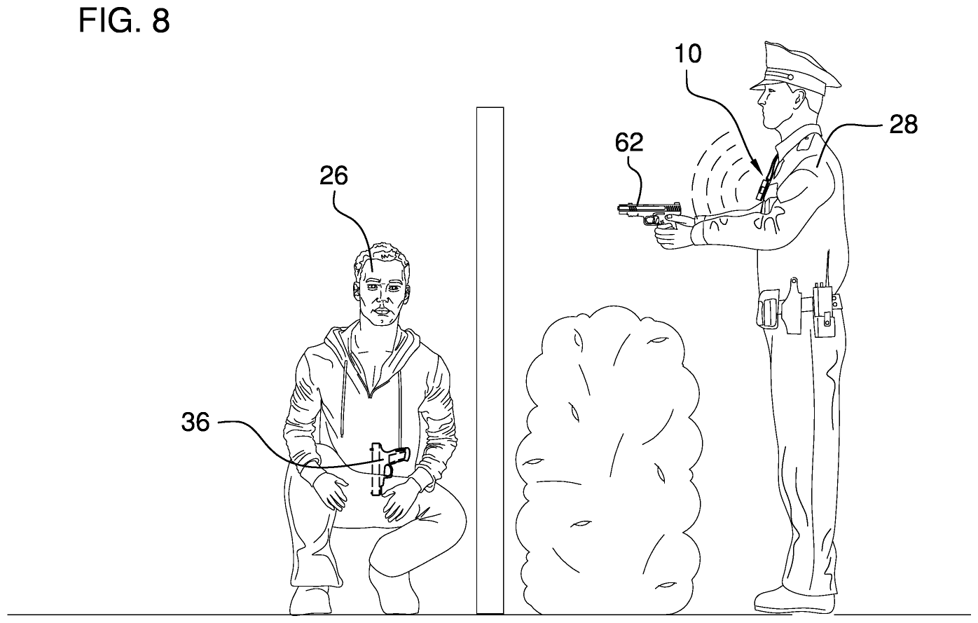

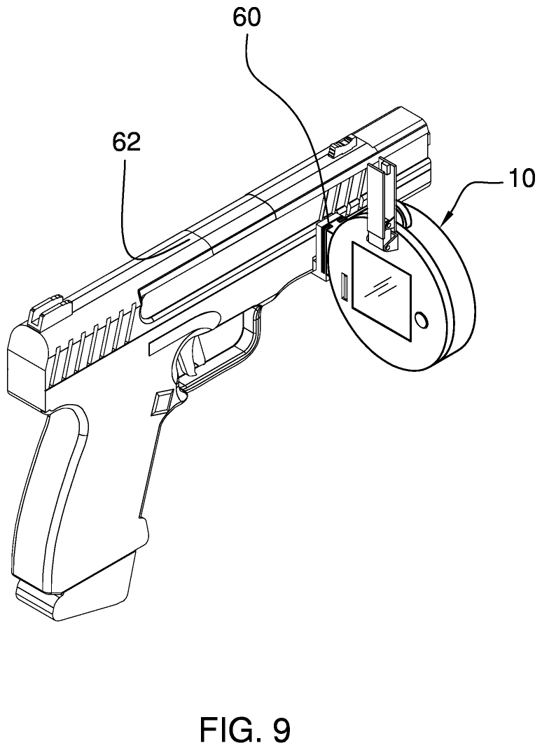

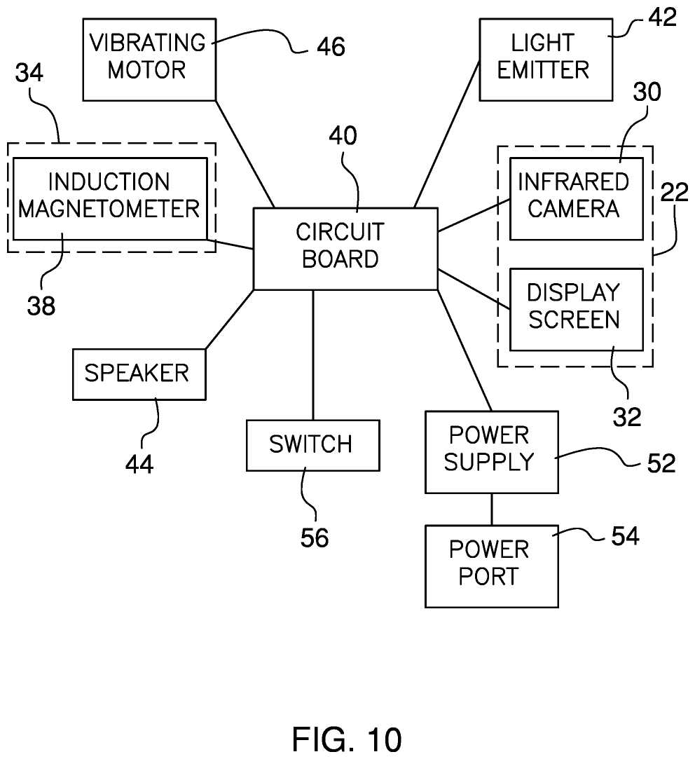

With reference now to the drawings, and in particular to FIGS. 1 through 10 thereof, a new thermal sensor device embodying the principles and concepts of an embodiment of the disclosure and generally designated by the reference numeral 10 will be described.

As best illustrated in FIGS. 1 through 10, the thermal and metal detection device 10 generally comprises a housing 12 having an exterior surface 14 and including a front wall 16, a back wall 18, and a perimeter wall 20. The perimeter wall 20 is positioned between the front wall 16 and the back wall 18, and the housing 12 has a cylindrical configuration. Alternatively, the housing 12 may have a rectangular configuration, a cubic configuration, or any other geometrical configuration. An infrared sensor 22 is coupled to the housing 12 and is configured to detect a thermal signature 24 of a person 26 within a vicinity of a user 28. The user 28 may be pursuing the person 26, such as a law enforcement officer pursuing a suspect or a firefighter searching for people within a building.

The infrared sensor 22 includes an infrared camera 30 mounted on the exterior surface 14 of the front wall 16. The infrared camera 30 emits infrared radiation outwardly from the front wall 16 to detect and measure surface temperatures. The infrared camera 30 produces virtual images configured to show surface temperature on objects detected by the infrared camera 30. A display screen 32 is mounted on the exterior surface 14 of the back wall 18. The infrared camera 30 is electrically coupled to the display screen 32, wherein the display screen 32 displays virtual images from the infrared camera 30. The virtual images displayed on the display screen 32 may facilitate locating the person 26 being pursued by the user 28.

A metal sensor 34 is coupled to the housing 12 and is configured to detect a metal object 36 within the vicinity of the user 28. The metal sensor 34 is an induction magnetometer 38 mounted to the exterior surface 14 of the front wall 16 and is configured to detect a magnetic flux from a metal object 36. The metal object 36 may include a metal weapon attached to the person 26 being pursued by the user 28. A circuit board 40 is electrically coupled to the infrared sensor 22 and to the metal sensor 34. The circuit board 40 is positioned within the housing 12. A light emitter 42 is electrically coupled to the circuit board 40 and is actuated by the circuit board 40 to emit light when the infrared sensor 22 is configured to detect the thermal signature 24 of a person 26 or when the metal sensor 34 is configured to detect the metal object 36. The light emitter 42 is mounted on the exterior surface 14 of the front wall 16. The light emitter 42 is a light emitting diode that may emit light continuously or periodically when actuated.

A speaker 44 is electrically coupled to the circuit board 40 is actuated by the circuit board 40 the to emit an audible alarm when the infrared sensor 22 is configured to detect the thermal signature 24 of the person 26 or when the metal sensor 34 is configured to detect the metal object 36. The speaker 44 is mounted on the exterior surface 14 of the front wall 16. The speaker 44 may be actuated when the infrared sensor 22 detects the thermal signature 24 of the person 26 and the light emitter 42 may be actuated when the metal sensor 34 detects the metal object 36. Alternatively, the light emitter 42 may be actuated when the infrared sensor 22 detects the thermal signature 24 of the person 26 and the speaker 44 may be actuated when the metal sensor 34 detects the metal object 36.

A vibrating motor 46 is electrically coupled to the circuit board 40 and is actuated by the circuit board 40 to emit vibrations when the infrared sensor 22 is configured to detect the thermal signature 24 of the person 26 or when the metal sensor 34 is configured to detect the metal object 36. The vibrating motor 46 is positioned within the housing 12. The vibrating motor 46 typically includes a weighted member 48 attached to a shaft 50 extending outwardly from the vibrating motor 46. The vibration motor is turned on to rotate the shaft 50 wherein the weighted member 48 produces vibrations when the shaft 50 rotates.

A power supply 52 is electrically coupled to the circuit board 40 and is positioned within the housing 12. The power supply 52 is a rechargeable battery, and a power port 54 is electrically coupled to the power supply 52. The power port 54 is configured to receive a power cord, wherein the power cord provides electricity to the power supply 52. The power port 54 comprises a universal serial bus port positioned on the back wall 18 of the housing 12. A switch 56 is electrically coupled to the circuit board 40 and is actuated to turn on or off the infrared sensor 22 and the metal sensor 34. The switch 56 comprises a push button mounted on the exterior surface 14 of the back wall 18.

A plurality of securing members 58 is coupled to the housing 12. Each of the securing members 58 is configured to secure the housing 12 to an object proximate to the user 28 such that only one of the securing members 58 selectively secures the housing 12 to the object when in-use. The securing members 58 include a firearm mount 60 coupled to the housing 12. The firearm mount 60 is configured to mount the housing 12 to a firearm 62. The firearm mount 60 is positioned on a first side section of the perimeter wall 20, wherein the metal sensor 34 and the infrared sensor 22 are configured to face forwardly from the firearm 62 when the housing 12 is mounted to the firearm 62.

The securing members 58 include a fastener 64 coupled to the housing 12. The fastener 64 is configured to removably fasten the housing 12 to a garment 68 of the user 28. The fastener 64 comprises a clip pivotally coupled to a pivot member 66 mounted on the exterior surface 14 of the back wall 18. The garment 68 of the user 28 may be a shirt or a belt, wherein the clip attaches to the shirt or the belt of the user 28. The securing members 58 include a loop connector 70 coupled to the housing 12. The loop connector 70 is configured to secure to a lanyard 72 worn about a neck of the user 28. The loop connector 70 is positioned on a top section of the perimeter wall 20 such that the housing 12 hangs downwardly when the loop connector 70 is attached to the lanyard 72 worn about the neck of the user 28.

In use, only one of the securing members 58 attaches to the housing 12 to the object proximate to the user 28, wherein the firearm mount 60 may mount the housing 12 to the firearm 62 or the fastener 64 may attach to the garment 68 of the user 28 or the loop connector 70 may attached to the lanyard 72 worn about the neck of the user 28. The switch 56 is engaged to turn on the infrared sensor 22 and the metal sensor 34. The infrared camera 30 of the infrared sensor 22 and the induction magnetometer 38 of the metal sensor 34 are pointed towards the vicinity where the user 28 would like to scan for the person 26 or the metal object 36. The light emitter 42, the speaker 44, and the vibrating motor 46 are actuated when the metal sensor 34 detects the magnetic flux of the metal object 36 or when the infrared sensor 22 detects the thermal signature 24 of the person 26.

With respect to the above description then, it is to be realized that the optimum dimensional relationships for the parts of an embodiment enabled by the disclosure, to include variations in size, materials, shape, form, function and manner of operation, assembly and use, are deemed readily apparent and obvious to one skilled in the art, and all equivalent relationships to those illustrated in the drawings and described in the specification are intended to be encompassed by an embodiment of the disclosure.

Therefore, the foregoing is considered as illustrative only of the principles of the disclosure. Further, since numerous modifications and changes will readily occur to those skilled in the art, it is not desired to limit the disclosure to the exact construction and operation shown and described, and accordingly, all suitable modifications and equivalents may be resorted to, falling within the scope of the disclosure. In this patent document, the word “comprising” is used in its non-limiting sense to mean that items following the word are included, but items not specifically mentioned are not excluded. A reference to an element by the indefinite article “a” does not exclude the possibility that more than one of the element is present, unless the context clearly requires that there be only one of the elements.

I claim:

1. A thermal detection device configured to alert a user when a person is proximate to the user, said thermal detection device comprising:
a housing having an exterior surface and including a front wall, a back wall, and a perimeter wall;
an infrared sensor being coupled to said housing, said infrared sensor being configured to detect a thermal signature of a person within a vicinity of the user;
a metal sensor being coupled to said housing, said metal sensor being configured to detect a metal object within the vicinity of the user;
a circuit board being electrically coupled to said infrared sensor and to said metal sensor;
a light emitter being electrically coupled to said circuit board, said light emitter being actuated by said circuit board to emit light when said infrared sensor is configured to detect the thermal signature of a person or when said metal sensor is configured to detect the metal object;
a speaker being electrically coupled to said circuit board, said speaker being actuated by said circuit board said to emit an audible alarm when said infrared sensor is configured to detect the thermal signature of a person or when said metal sensor is configured to detect the metal object;
a vibrating motor being electrically coupled to said circuit board, said vibrating being actuated by said circuit board to emit vibrations when said infrared sensor is configured to detect the thermal signature of a person or when said metal sensor is configured to detect the metal object;
a power supply being electrically coupled to said circuit board;
a switch being electrically coupled to said circuit board, said switch being actuated to turn on or off said infrared sensor and said metal sensor; and
a plurality of securing members being coupled to said housing, each of said securing members being configured to secure said housing to an object proximate to the user such that only one of said securing members selectively secures said housing to the object when in-use.

2. The thermal detection device of claim 1, wherein said perimeter wall is positioned between said front wall and said back wall, said housing having a cylindrical configuration.

3. The thermal detection device of claim 1, wherein said infrared sensor includes an infrared camera being mounted on said exterior surface of said front wall, said infrared camera emitting infrared radiation outwardly from said front wall to detect and measure surface temperatures, said infrared camera producing virtual images configured to show surface temperature on objects detected by said infrared camera.

4. The thermal detection device of claim 3, further including a display screen being mounted on said exterior surface of said back wall, said infrared camera being electrically coupled to said display screen, wherein said display screen displays virtual images from said infrared camera.

5. The thermal detection device of claim 1, wherein said metal sensor is an induction magnetometer mounted to said exterior surface of said front wall and is configured to detect a magnetic flux from a metal object.

6. The thermal detection device of claim 1, wherein said circuit board is positioned within said housing.

7. The thermal detection device of claim 1, wherein said light emitter is mounted on said exterior surface of said front wall.

8. The thermal detection device of claim 1, wherein said speaker is mounted on said exterior surface of said front wall.

9. The thermal detection device of claim 1, wherein said vibrating motor is positioned within said housing.

10. The thermal detection device of claim 1, wherein said power supply is positioned within said housing, said power supply being a rechargeable battery.

11. The thermal detection device of claim 10, further including a power port being electrically coupled to said power supply, said power port being configured to receive a power cord, wherein said power cord provides electricity to said power supply, said power port comprising a universal serial bus port positioned on said back wall of said housing.

12. The thermal detection device of claim 1, wherein said switch comprises a push button mounted on said exterior surface of said back wall.

13. The thermal detection device of claim 1, wherein said securing members includes a firearm mount being coupled to said housing, said firearm mount being configured to mount said housing to a firearm, said firearm mount being positioned on a first side section of said perimeter wall, wherein said metal sensor and said infrared sensor are configured to face forwardly from the firearm when said housing is mounted to the firearm.

14. The thermal detection device of claim 13, further including a fastener being coupled to said housing, said fastener being configured to removably fasten said housing to a garment of the user, said fastener comprising a clip, said clip being pivotally coupled to a pivot member mounted on said exterior surface of said back wall.

15. The thermal detection device of claim 13, further including a loop connector being coupled to said housing, said loop connector being configured to secure to a lanyard worn about a neck of the user, said loop connector being positioned on a top section of said perimeter wall.

16. A thermal detection device configured to alert a user when a person is proximate to the user, said thermal detection device comprising:
a housing having an exterior surface and including a front wall, a back wall, and a perimeter wall, said perimeter wall being positioned between said front wall and said back wall, said housing having a cylindrical configuration;
an infrared sensor being coupled to said housing, said infrared sensor being configured to detect a thermal signature of a person within a vicinity of the user, said infrared sensor including:
an infrared camera being mounted on said exterior surface of said front wall, said infrared camera emitting infrared radiation outwardly from said front wall to detect and measure surface temperatures, said infrared camera producing virtual images configured to show surface temperature on objects detected by said infrared camera; and
a display screen being mounted on said exterior surface of said back wall, said infrared camera being electrically coupled to said display screen, wherein said display screen displays virtual images from said infrared camera;
a metal sensor being coupled to said housing, said metal sensor being configured to detect a metal object within the vicinity of the user, said metal sensor being an induction magnetometer mounted to said exterior surface of said front wall and being configured to detect a magnetic flux from a metal object;
a circuit board being electrically coupled to said infrared sensor and to said metal sensor, said circuit board being positioned within said housing;
a light emitter being electrically coupled to said circuit board, said light emitter being actuated by said circuit board to emit light when said infrared sensor is configured to detect the thermal signature of a person or when said metal sensor is configured to detect the metal object, said light emitter being mounted on said exterior surface of said front wall;
a speaker being electrically coupled to said circuit board, said speaker being actuated by said circuit board said to emit an audible alarm when said infrared sensor is configured to detect the thermal signature of a person or when said metal sensor is configured to detect the metal object, said speaker being mounted on said exterior surface of said front wall;
a vibrating motor being electrically coupled to said circuit board, said vibrating being actuated by said circuit board to emit vibrations when said infrared sensor is configured to detect the thermal signature of a person or when said metal sensor is configured to detect the metal object, said vibrating motor being positioned within said housing;
a power supply being electrically coupled to said circuit board, said power supply being positioned within said housing, said power supply being a rechargeable battery;
a power port being electrically coupled to said power supply, said power port being configured to receive a power cord, wherein said power cord provides electricity to said power supply, said power port comprising a universal serial bus port positioned on said back wall of said housing;
a switch being electrically coupled to said circuit board, said switch being actuated to turn on or off said infrared sensor and said metal sensor, said switch comprising a push button mounted on said exterior surface of said back wall;
a plurality of securing members being coupled to said housing, each of said securing members being configured to secure said housing to an object proximate to the user such that only one of said securing members selectively secures said housing to the object when in-use, said securing members including:
a firearm mount being coupled to said housing, said firearm mount being configured to mount said housing to a firearm, said firearm mount being positioned on a first side section of said perimeter wall, wherein said metal sensor and said infrared sensor are configured to face forwardly from the firearm when said housing is mounted to the firearm;
a fastener being coupled to said housing, said fastener being configured to removably fasten said housing to a garment of the user, said fastener comprising a clip, said clip being pivotally coupled to a pivot member mounted on said exterior surface of said back wall; and
a loop connector being coupled to said housing, said loop connector being configured to secure to a lanyard worn about a neck of the user, said loop connector being positioned on a top section of said perimeter wall.
